(19) United States
(12) Patent Application Publication
(10) Pub. No.: US 2014/0315855 A1
(43) Pub. Date: Oct. 23, 2014

(54) Antidandruff Agent Sulfated Polysaccharides

(71) Applicant: L'OREAL, Paris (FR)

(72) Inventors: Anne Potter, Neuilly-Sur-Seine (FR); Sebastien Thibaut, Puteaux (FR); Christele Ribaut, Nogent S/Marne (FR)

(21) Appl. No.: 14/365,297

(22) PCT Filed: Dec. 14, 2012
(86) PCT No.: PCT/FR2012/052936

§ 371 Date: Jun. 13, 2014

(30) Foreign Application Priority Data
Dec. 20, 2011 (FR) 1162109

(51) Int. Cl. A61K 31/737

(52) U.S. Cl. CPC A61K 31/737
USPC 514/59; 514/54

(57) ABSTRACT

The invention relates to the cosmetic use of a sulfated polysaccharide chosen from: dextran sulfate and sulfated polysaccharides derived from the red alga Porphyridium sp, as active agent for preventing and/or treating dandruff of the scalp.

The present invention relates to the use of particular sulfated polysaccharides as an anti-dandruff agent, in particular in the cosmetic treatment of dandruff conditions associated with the excessive proliferation of yeasts of the Malassezia genus on the scalp. The invention also relates to a cosmetic treatment process intended to eliminate and/or reduce dandruff, in particular that caused by yeasts of the Malassezia genus, which employs said sulfated polysaccharides.

The occurrence of dandruff, corresponding to a desquamative disorder of the scalp, is disagreeable both esthetically and because of the unpleasantness that it causes (itching, redness, etc.), so many people confronted with this problem to varying degrees wish to eliminate it efficiently and permanently.

Dandruff corresponds to excessive and visible desquamation of the scalp resulting from excessively rapid multiplication of the epidermal cells. This phenomenon may be caused in particular by microtraumas of physical or chemical nature, such as excessively aggressive hair treatments, extreme climatic conditions, nervousness, the diet, fatigue and pollution, but it has been demonstrated that dandruff conditions usually result from a disorder of the microflora of the scalp and are more particularly due to the excessive colonization of a yeast belonging to the family of yeasts of the Malassezia genus (previously known as Pytirosporum ovale) and which is naturally present on the scalp.

For combating dandruff, it is known practice to use topically applied antifungal agents. By virtue of their antifungal capacity, these agents aim to eliminate or control the multiplication of a resident yeast of the scalp, belonging to the Malassezia genus and its variants (M. ovalis, M. orbiculare, M. furfur, M.globosa, etc.).

Numerous agents are claimed, known and used to this effect, among which mention may be made of zinc pyrithione, piroctone olamine and selenium disulfide. However, these antidandruff agents are not completely satisfactory in terms of effectiveness (immediate effectiveness or duration of the effect) and/or in terms of impact on the environment.

The objective of the present invention is to provide antidandruff agents which are nonirritant to the skin and the scalp, and which are as effective as the known antidandruff agents, while at the same time having a more favorable environmental impact (low bioaccumulation and good biodegradability in particular).

The objective of the invention is also to provide active agents which make it possible to prevent excessive colonization of the scalp by Malassezia sp, and in particular to limit the desquamative disorder associated with the metabolism of Malassezia.

The applicant has now found, surprisingly, that the use of of certain sulfated polysaccharides as defined hereinafter makes it possible to effectively treat dandruff conditions, in particular those associated with the proliferation of yeasts of the Malassezia genus, and to remedy the drawbacks of the prior art.

It has been observed that, by employing the sulfated polysaccharides defined hereinafter, it is possible to eliminate and/or reduce the number of dandruff flakes, and the desquamative disorder created by the Malassezia and their metabolites, such as oleic acid.

A subject of the present invention is therefore the cosmetic use of particular sulfated polysaccharides as defined hereinafter, as an active agent for preventing and/or treating dandruff of the scalp.

A subject of the invention is also a cosmetic treatment process for preventing and/or treating dandruff, in particular that caused by yeasts of the Malassezia genus, characterized in that it comprises the application, to the scalp, of at least one sulfated polysaccharide as defined hereinafter.

Advantageously, the use and the process according to the invention are implemented for treating dandruff due to the proliferation of yeasts of the Malassezia genus on the scalp.

The sulfated polysaccharides used according to the invention are chosen from dextran sulfate and the sulfated polysaccharides derived from the red alga Porphyridium sp.

Advantageously, the degree of sulfation of the polysaccharides may range from 1% to 30% by weight, relative to the weight of the polysaccharide. Preferably, this degree of sulfation may range from 2% to 25% by weight.

The polysaccharide may optionally be acetylated. The degree of acetylation may range from 0 to 10% by weight (degree by weight of acetylated units relative to the total weight of the polymer).

The polysaccharide used according to the invention preferably has a weight-average molecular weight of between 1000 and 20×10⁶ Daltons, preferentially between 2000 and 10×10⁶ Da, preferentially between 5000 and 5×10⁶ Da and more preferentially between 10 000 and 1×10⁶ Da.

The sulfated polysaccharide used according to the invention may be dextran sulfate. The dextran sulfate sold under the trade name “Dextran sulfate 10 sodium salt CG” by the company PK chemicals may be used.

The sulfated polysaccharide used according to the invention may be chosen from the sulfated polysaccharides derived from the red alga Porphyridium sp, such as the product sold under the name “Alguard®” by the company Frutarom. Application EP-A-311496 describes in particular a process for preparing such polysaccharides. These sulfated polysaccharides contain glucuronic acid, xylose, glucose and galactose which are sulfated.

The sulfated polysaccharide used according to the invention is advantageously present in a cosmetic composition containing a physiologically acceptable medium. The term “physiologically acceptable medium” is intended to mean a medium that is compatible with cutaneous tissues such as the skin and the scalp.

The sulfated polysaccharide used according to the invention may be present in the composition in a content ranging from 0.01% to 20% by weight, relative to the total weight of the cosmetic composition, preferably ranging from 0.05% to 15% by weight, preferentially ranging from 0.1% to 10% by weight and more preferentially ranging from 0.1% to 5% by weight.

The physiologically acceptable medium of the composition may more particularly consist of water and optionally of a physiologically acceptable organic solvent chosen, for example, from lower alcohols containing from 2 to 8 carbon atoms and in particular from 2 to 6 carbon atoms, for instance ethanol, isopropanol, propanol or butanol; polyethylene glycols containing from 6 to 80 ethylene oxide units, and polyols, for instance propylene glycol, isoprene glycol, butylene glycol, glycerol and sorbitol.

The compositions according to the invention may be in all the galenical forms conventionally used for topical application and in particular in the form of aqueous or aqueous-alcoholic solutions, of oil-in-water (O/W), water-in-oil (W/O) or multiple (triple: W/O/W or O/W/O) emulsions, of aqueous gels or of dispersions of a fatty phase in an aqueous phase using spherules, it being possible for these spherules to be polymeric nanoparticles, such as nanospheres and nanocapsules, or lipid vesicles of ionic and/or nonionic type (liposomes, niosomes or oleosomes). These compositions are prepared according to the usual methods.

In addition, the compositions used according to the invention may be more or less fluid and may have the appearance of a white or coloured cream, a pomade, a milk, a lotion, a serum, a paste, a mousse or a shampoo.

The composition used according to the invention comprises adjuvants usually employed in the cosmetics field, and in particular chosen from water; oils; waxes, pigments, fillers, dyes, surfactants, emulsifiers; cosmetic active agents, UV-screening agents, polymers, thickeners, film-forming polymers, preservatives, fragrances, bactericides, odor absorbers and antioxidants.

The amounts of these various adjuvants are those conventionally used in the field under consideration, and are for example from 0.01% to 20% of the total weight of the composition.

The examples hereinafter illustrate the invention without, however, limiting the scope thereof.

### EXAMPLE 1

### Evaluation of the Antidandruff Action of the Sulfated Polysaccharides

### Protocol

Demonstration of the Regulation of Epidermal Homeostasis by the Sulfated Polysaccharides on a Model of Reconstructed Skin Modified with a Scalp Flora Metabolite

The effect of the application of sulfated polysaccharides was evaluated in a reconstructed skin model exhibiting a cutaneous homeostasis disorder generating the appearance of aggregates analogous to dandruff.

This disorder is obtained by the topical application (for 48 hours) of a propylene glycol/ethanol/water solution (proportions by volume 5/60/35) containing 5% by weight of oleic acid. This method is described in the literature for causing the formation of dandruff on the surface of human skin (Y. M. DeAngelis et al., Three Etiologic Facets of Dandruff and Seborrheic Dermatitis: Malassezia Fungi, Sebaceous Lipids, and Individual Sensitivity. J Investig Dermatol Symp Proc 10:295-297, 2005). In the same way, application of oleic acid to samples of reconstructed skin brings about an imbalance in cutaneous homeostasis (cell hyperproliferation and lack of terminal differentiation) and causes the formation of surface aggregates called “dandruff-like flakes” similar to the dandruff observed on a scalp.

a) Reception of Skins

The reconstructed skins, produced by the company Episkin, are provided as plates containing a gelled growth medium (GDA3F from Episkin). On reception, the skins were placed in new “deep-well” dishes containing 8.5 ml of (liquid) GDA3F medium, and then placed in an incubator at 37° C., the atmosphere of which is saturated with water and CO₂-controled (5%).

b) 5 Conditions studied on skin:
1 “control” skin: the skin has received no treatment
1 “vehicle control” skin
2 skins “modified with oleic acid”
2 “sulfated polysaccharide control” skins
2 skins “modified with oleic acid +application of sulfated polysaccharide”

c) Treatments

After 20 days: Renewal of all the media (8.5 ml)
The “control” skin: the skin received no treatment. 25 μl of solutions were applied to each of the other skins. The vehicle medium is a mixture of propylene glycol/ethanol/water (5/60/35). The sulfated polysaccharide is applied at 2% by weight in the vehicle medium.

After 22 days: The tests were stopped.
Specimens were taken of the various skin samples. The skin samples were photographed using a Nikon 5000D apparatus, then samples were taken using a notch press (punch 12 mm in size cut out) and then embedded in tissue-TEK.

The experiment was repeated on three batches of different skins.

d) Methods of Analysis of the Results and Scoring

Macroscopic Observations of the Surface of the Samples and Scoring of the Dandruff-Like Condition of the Reconstructed Skin

In order to evaluate the surface condition of the samples, photographed reconstructed skins were observed and the dandruff-like condition was scored using a measurement scale (table below) defining the dandruff-like condition according to the surface of the sample covered with the dandruff-like flakes.

| Surface condition observed | Score assigned |
|---|---|
| Surface intact | 0 |
| Surface covered with a few | 1 |
| dandruff-like areas | |
| More than half the surface | 2 |
| covered with dandruff-like flakes | |
| Surface totally covered with | 3 |
| dandruff-like flakes | |

### RESULTS

### EXAMPLE 1a

### Sulfated Polydextran

The polysaccharide tested is dextran sulfate, sold under the trade reference “Dextran sulfate 10 sodium salt CG” by the company PK Chemicals.

| | Dandruff-like |
|---|---|
| Conditions tested on skin | score |
| Control | Score 0 |
| Vehicle Control | Score 0 |
| Sulfated Polydextran Control | Score 0 |
| Oleic Acid Application | Score 3 |
| Sulfated Polydextran Application + Oleic Acid | Score 0 |
| Application | |

### EXAMPLE 1b

### Polysaccharide Derived from Porphyridium

The polysaccharide derived from Porphyridium is sold under the name Alguard® by the company Frutarom.

| | Dandruff-like |
|---|---|
| Conditions tested on skin | score |
| Control | Score 0 |
| Vehicle Control | Score 0 |
| Alguard Porphyridium Control | Score 0 |
| Oleic Acid Application | Score 3 |
| Alguard Porphyridium Application + Oleic Acid | Score 0 |
| Application | |

### EXAMPLE 1c:

### Carrageenan (Outside the Invention)

Two different carrageenans were evaluated:

Carrageenan 1: sold under the name Satiagum UTC 30 by the company Cargill

Carrageenan 2: sold under the name Satiagum UTC 10 by the company Cargill

| | Dandruff-like |
|---|---|
| Conditions tested | score |
| Control | Score 0 |
| Vehicle Control | Score 0 |
| Carrageenan 1 Control | Score 0 |
| Oleic Acid Application | Score 3 |
| Carrageenan 1 Application + Oleic Acid | Score 1 |
| Application | |

| | Dandruff-like |
|---|---|
| Conditions tested | score |
| Control | Score 0 |
| Vehicle Control | Score 0 |
| Carrageenan 2 Control | Score 0 |
| Oleic Acid Application | Score 3 |
| Carrageenan 2 Application + Oleic Acid | Score 2 |
| Application | |

### CONCLUSIONS

The results obtained show that treatment of the skin with dextran sulfate or the polysaccharide derived from Porphyridium has an effective antidandruff action: no dandruff-like flake is observed at the surface of the skin samples treated with these sulfated polysaccharides.

The application of carrageenans 1 and 2 does not make it possible to totally inhibit the appearance of dandruff-like flakes at the surface of the treated reconstructed skin samples. They do not have an effective antidandruff action.

### EXAMPLE 2

### Determination of the Absence of Antifungal Activity on the Malassezia Strain

Preparation of the solution of sulfated polysaccharide tested at 1% by weight in modified Leeming and Notman (LNm) agar.

The initial solutions of the products to be tested are twice as concentrated as the final test concentration so as to take into account the dilution to ½ when bringing into contact with the suspension of Malassezia.

The test is carried out on the Malassezia globosa and Malassezia restricta strains.

Bring the Malassezia strains into contact according to the table below:

| | Growth control | Test Solution |
|---|---|---|
| Malassezia Strain | 0.5 ml | 0.5 ml |
| Sulfated polysaccharide | | 0.5 ml |
| solution | | |
| LNm | 0.5 ml | |

Stir and deposit the mixture at the surface of the LNm agar. Spread with a sterile scraper over the entire surface before recovering the excess. Incubate for at least 5 days at 30° C.

The antifungal effect of the sulfated polysaccharide tested was evaluated by means of the absence of growth of the Malassezia strain tested, relative to the growth control. The inhibitions were graded from 3 to 0 by assessing the density of the culture at the surface of the agar in comparison with the growth control of the strain.

| GRADING | INHIBITION | INTERPRETATION |
|---|---|---|
| 3 | 100% | No growth |
| 2 | 75% | Growth < control dish |
| 1 | 25% | Growth < control dish |
| 0 | 0% | Growth comparable to control dish |

The following results were obtained:

| | | Malassezia | Malassezia |
|---|---|---|---|
| % Inhibition | | restricta | globosa |
| Growth control | | Dense culture | Dense culture |
| Alguard ® | 1% | 0 | 0 |

The results obtained show that the polysaccharide tested (Alguard®) does not exhibit an antifungal activity on the Malassezia globosa and restricta species.

### EXAMPLE 3

### Antidandruff Shampoo

An antidandruff shampoo comprising the following ingredients is prepared:

| | | |
|---|---|---|
| Sodium lauryl ether sulfate (2.2 OE) in aqueous | 17 | g AM |
| solution | | |
| (Texapon AOS 225 UP from Cognis) | | |
| Cocoylbetaine in aqueous solution | 2.5 | g AM |
| (Dehyton AB 30 from Cognis) | | |
| Coconut acid monoisopropanolamide (Rewomid V | 2.0 | g |
| 3203 from Goldschmidt) | | |
| Dextran sulfate | 0.3 | g AM |
| Preservatives | 1.1 | g |

| | |
|---|---|
| Fragrance | 0.5 |
| Water | QS 100 g |

The shampoo applied to the hair and the scalp makes it possible to reduce the appearance of dandruff.

### EXAMPLE 4

### Antidandruff Lotion

An antidandruff lotion comprising the following ingredients is prepared:

| | |
|---|---|
| Sulfated polysaccharide derived from the red alga | 0.3 g AM |
| Porphyridium sp (“Alguard ®” from Frutarom) | |
| Preservatives | qs |
| Water | qs 100 g |

The lotion applied to the hair and the scalp makes it possible to reduce the appearance of dandruff.

1. (canceled)

2. (canceled)

3. (canceled)

4. (canceled)

5. A cosmetic treatment process intended to eliminate and/or reduce dandruff, which process comprises applying to the scalp, at least one sulfated polysaccharide selected form the group consisting of dextran sulfate and sulfated polysaccharides derived from the red alga Porphyridium sp, as an active agent or of a cosmetic composition containing said sulfated polysaccharide.

6. The process as claimed in claim 5, wherein the sulfated polysaccharide is present in a cosmetic composition in a content ranging from 0.01% to 20% by weight, relative to the total weight of the cosmetic composition.

7. The process as claimed in claim 6, wherein said dandruff is caused by yeasts of the Malassezia genus.

8. The process as claimed in claim 7, wherein the sulfated polysaccharide is dextran sulphate.

9. The process as claimed in claim 7, wherein the sulfated polysaccharide is derived from the red alga Porphyridium sp.

10. The process as claimed in claim 6, wherein the sulfated polysaccharide is dextran sulphate.

11. The process as claimed in claim 6, wherein the sulfated polysaccharide is derived from the red alga Porphyridium sp.

12. The process as claimed in claim 5, wherein said dandruff is caused by yeasts of the Malassezia genus.

13. The process as claimed in claim 12, wherein the sulfated polysaccharide is dextran sulphate.

14. The process as claimed in claim 12, wherein the sulfated polysaccharide is derived from the red alga Porphyridium sp.

15. The process as claimed in claim 5, wherein the sulfated polysaccharide is dextran sulphate.

16. The process as claimed in claim 5, wherein the sulfated polysaccharide is derived from the red alga Porphyridium sp.
